(19) United States
(12) Patent Application Publication
(10) Pub. No.: US 2006/0020217 A1
(43) Pub. Date: Jan. 26, 2006

(54) Wireless self-adhesive heartbeat detector

(76) Inventor: Chin-Shu Lin, Tainan Country (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/873,245

(22) Filed: Jun. 23, 2004

(51) Int. Cl. A61B 5/04

(52) U.S. Cl. 600/509

(57) ABSTRACT

A wireless self-adhesive heartbeat detector includes two terminals with one positive pole and one negative pole. The detector comprises a glue layer to attach to a human body so as to measure the heartbeat of the person. This design does not use any traditional string or tape, thus the user does not have any uncomfortable feeling.

### BACKGROUND OF THE INVENTION

1. Field of the Invention

This invention relates to a wireless self-adhesive heartbeat detector, and more particularly to a detector adhesive to human's chest without using any string or tape.

2. Description of the Prior Art

There are plenty of heartbeat detectors widely used by medical groups and health centers. They are designed from wire to wireless for its convenience. As shown in FIG. 6, a detector A extends towards respective sides to form a pair of detecting sections A1 with a detecting terminal A11, and a pair of buckles A12 at the respective far ends. A belt B comprises a pair of buckles B1 to be secured with the buckles A12 and to fasten the detector A1 to the chest of the human with the two detecting sections A1 engaging with the skin of the chest and monitoring the heartbeat. The detector A will send a signal to an exterior receiver C.

The belt B restraining the chest makes the user uncomfortable, especially to female who has a large size chest, which makes the detector harder to be placed at the right position.

In view of these and many others, the inventor has invented the present invention to improve the shortcomings.

### SUMMARY OF THE INVENTION

It is the primary objective of the present invention to provide a wireless self-adhesive heartbeat detector, which is easy to attach to a human's chest and to measure the accuracy heartbeat.

It is another objective of the present invention to provide a wireless self-adhesive heartbeat detector, which is comfortable to users.

It is a further objective of the present invention to provide a wireless self-adhesive heartbeat detector, which is easy to operate.

### BRIEF DESCRIPTION OF THE DRAWINGS

FIG. 1 is an exploded view of the present invention;

FIG. 2 is a side view of the present invention, partially sectioned;

FIG. 3 is a view showing a man in exercise with the present invention attached to the chest to measure the heartbeat;

FIG. 4 is an exploded view of a second embodiment of the present invention;

FIG. 5 is an exploded view of a third embodiment of the present invention; and

FIG. 6 is a prior art of a heartbeat detector in use.

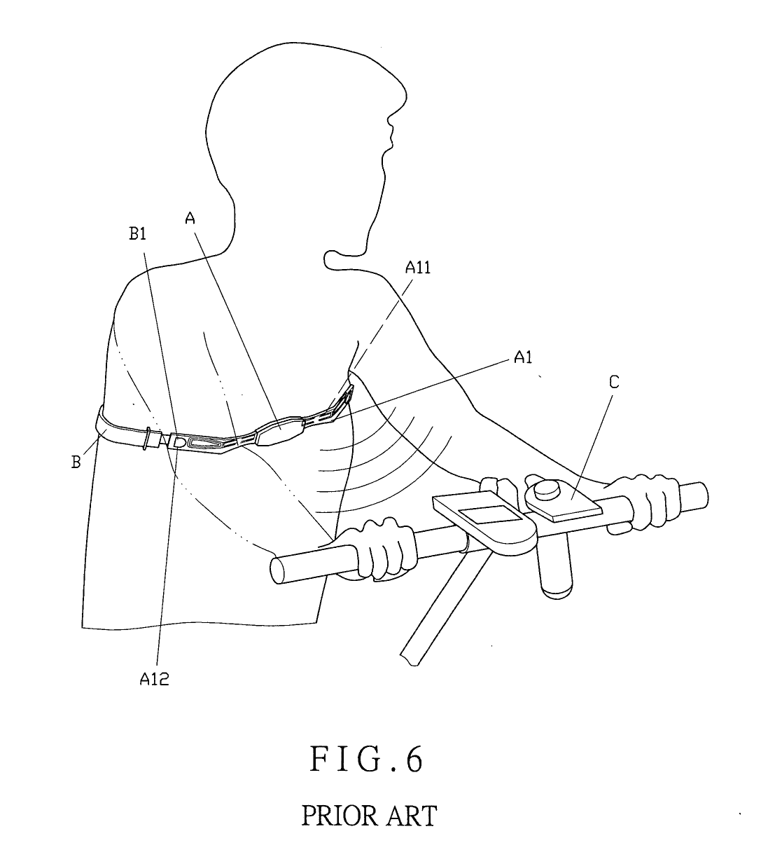

### DETAILED DESCRIPTION OF THE PREFERRED EMBODIMENT

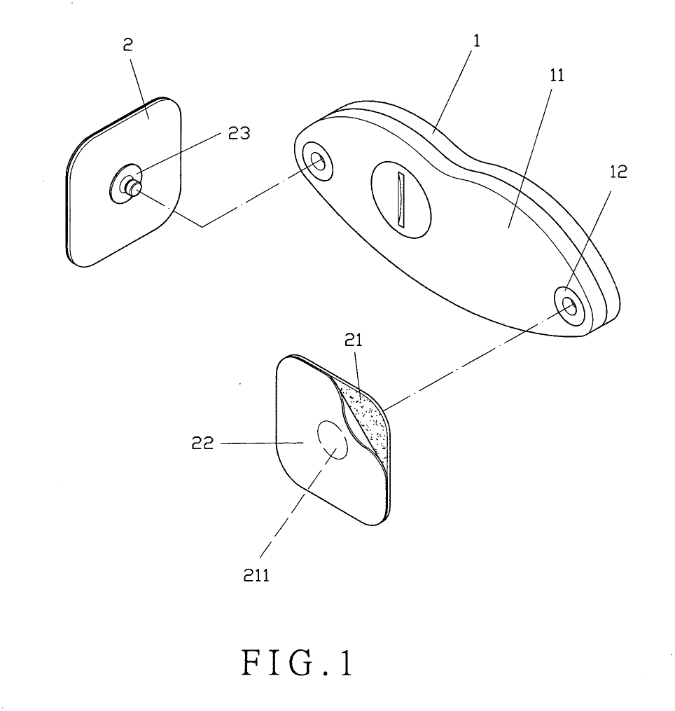

As shown in FIG. 1, the present invention comprises a detector 1 and a pair of stickers 2.

The detector 1 adopts a wireless method to send a signal of a human's heartbeat to a monitor and comprises a pair of terminals 12 on a contact surface 11. The terminals 12 are formed either denting inwardly or protruding outwardly. One of the terminals 12 is a positive pole, while the other is a negative pole.

Each sticker 2 has a glue layer 21 at one side surface with a pole end 211 and a connecting part 23 extending from the opposite end of the pole end 211. The glue layer 21 is covered by paper 22, when not in use. The connecting part 23 will extend into a corresponding terminal 12 when the sticker 2 is attached to the detector 1.

To measure a human's heartbeat, the paper 22 of each sticker 2 is torn off and place the stickers 2 against the chest with the two terminals 12 close to heart to receive the heartbeat attach

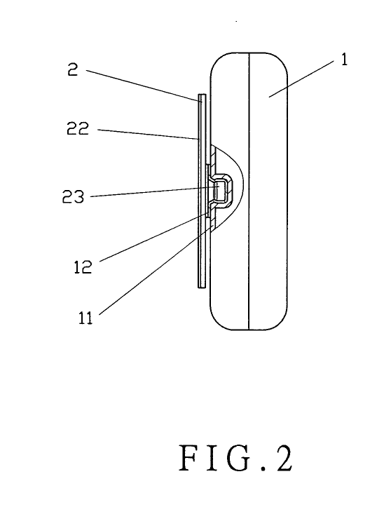

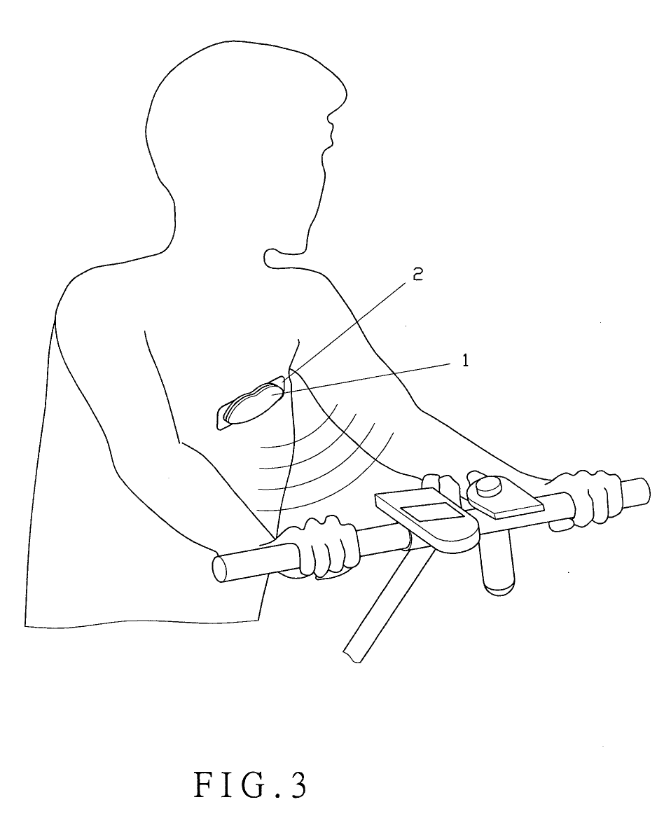

To assemble the present invention, as shown in FIGS. 2 and 3, the stickers 2 are attached to the detector 1 with the connecting parts 23 buckling into the terminals 12. The paper 22 of each sticker 2 is torn off, and then the detector 1 is attached to a human's chest. The softness of the stickers 2 will fit the human's body in such a way that the human will not feel any uncomfortable, whereas the pole ends 211 of the stickers 2 will detect the heartbeat and send to the detector 1 through the connecting parts 23.

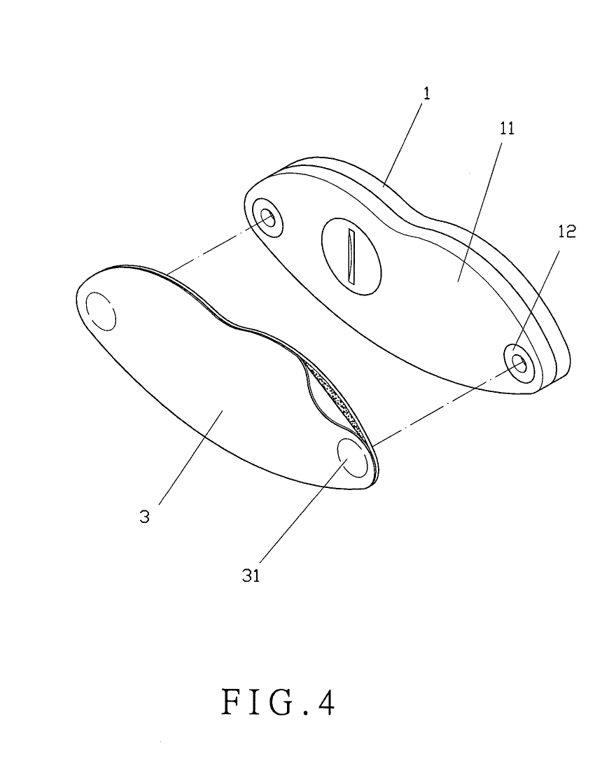

A sticker 3 may be formed in a one-piece relative to the detector 1. The sticker 3 comprises two pole ends 31 and two connecting parts (not shown in the drawing) integrally, as shown in FIG. 4. The sticker 3 is in a disposal form and can be discarded after each use, which corresponds to the sanitary requirement.

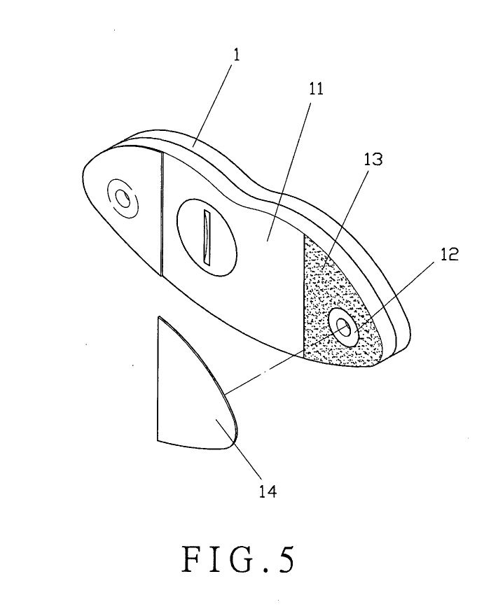

Further, the heartbeat detector can be formed as shown in FIG. 5, which forms a pair of glue layers 13 direct to the surface 11 of the detector 1. Each glue layer 13 is covered by paper 14. The heartbeat detector 1 of this design may be in a soft material to match the figure of the human's chest for comfort consideration, while the function is well performed.

Still further, the heartbeat detector of the present invention uses a wireless transmitting method, which eliminates the binding feeling from the design of a traditional heartbeat detector.

I claim:

1. A wireless self-adhesive heartbeat detector comprising a pair of terminals with one positive pole and one negative pole, said detector comprising a glue layer at one side.

2. The wireless self-adhesive heartbeat detector, as recited in claim 1, wherein said glue layer is formed direct to one surface of said detector.

3. The wireless self-adhesive heartbeat detector, as recited in claim 1, wherein said glue layer is attached to one surface of a sticker, said sticker comprising two pole ends at one side and two connecting parts at an opposite side to be connected with said terminals of said detector.

4. The wireless self-adhesive heartbeat detector, as recited in claim 3, wherein said sticker is formed into two separated pieces.

5. The wireless self-adhesive heartbeat detector, as recited in claim 3, wherein said sticker is formed in a one-piece.
